(19) United States
(12) Patent Application Publication
(10) Pub. No.: US 2005/0009029 A1
(43) Pub. Date: Jan. 13, 2005

(54) Expression system

(76) Inventors: Christopher Carl Frye, Plainfield, IN (US); Charles Lee Hershberger, Greenfield, IN (US)

Correspondence Address: ELI LILLY AND COMPANY;PATENT DIVISION, P.O. BOX 6288, INDIANAPOLIS, IN, 46206-6288, US

(21) Appl. No.: 10/495,468

(22) PCT Filed: Dec. 3, 2002
(86) PCT No.: PCT/US02/36141

Related U.S. Application Data
(60) Provisional application No. 60/339,676, filed on Dec. 12, 2001.

(51) Int. Cl. C12Q 1/68; C07H 21/04; C12N 1/20; C12N 9/22; C12N 15/86

(52) U.S. Cl. 435/6; 435/69.1; 435/456; 435/252.3; 435/199; 530/399; 536/23.2

(57) ABSTRACT

The present invention provides an expression system for producing a target protein in a host cell comprising a homologously integrated gene encoding T7 RNA polymerase, and a nonintegrated gene encoding a target protein.

The present invention relates to a novel host cell useful in an expression system for producing target proteins.

Many expression systems are available for the purpose of producing target proteins in bacterial host cells. Many of these systems are derived from naturally occurring endogenous regulatory systems like the lactose (lac) and tryptophan (trp) operons of E. coli. There are also several systems that utilize components of phage expression regulatory networks like the Lambda promoter (P_{L}) system of Lambda phage.

However, among the most widely and routinely used systems for expression of recombinant target proteins in E. coli at the laboratory level is a bacteriophage T7 expression system. The expression system is commercially available from Novagen, Inc. (Madison Wis.) and is described in U.S. Pat. No. 4,952,496. The expression system comprises a host cell comprising an integrated phage lysogen. The host cell is then transformed with a nonintegrated gene under control of a phage promoter, wherein the nonintegrated gene encodes a target protein of choice.

Lambda DE3 lysogen is a recombinant phage that carries a clone of T7 RNA polymerase under control of lacUV5 promoter. Lambda DE3 lysogens are prepared by co-infecting a host cell with a Lambda DE3 phage lysate, a helper phage lysate, and a selection phage lysate. The result of the co-infection is a host cell that has the Lambda DE3 phage incorporated into the host cells' chromosome. Although the Lambda DE3 phage is integrated into the host chromosome at the Lambda integration site, the Lambda DE3 phage is defective in its ability to be lytic. Thus, the DE3 lysogen should be stable and should not subsequently lyse the cells to produce infectious phage. Upon induction of the expression system, the host cells make T7 RNA polymerase from the DE3 lysogen. The T7 RNA polymerase then binds the phage promoter of the nonintegrated target gene and initiates synthesis of the target protein.

A T7 expression system provides many benefits that make it quite suitable to express target proteins. For example, the T7 or T7lac promoter of the target gene is a phage promoter that is unique to phage and is not recognized by the host cell RNA polymerases. Thus, expression of the target protein is only initiated when T7 RNA polymerase is present. This helps to reduce the potential for expression of the target protein prior to induction. Expression of the target protein prior to induction is not desirable because some target proteins have deleterious effects on host cell growth, thus, reducing maximum target protein production.

Another example that makes the T7 expression system suitable to express target proteins is the T7 promoter has been altered to include the lactose operator (lacO). The lacO is a binding site for the lactose operon repressor. The lactose repressor binds the lacO, which prevents the T7 RNA polymerase from binding the T7lac promoter, thus effectively repressing expression of the target protein. The repression is reversible upon addition of an inducing agent to the host cell. The inducing agent knocks the lactose repressor off the lacO and allows the T7 RNA polymerase to bind the T7lac promoter and initiate expression of the target protein. Inclusion of lacO tightens the initiation of expression of the target protein by nearly 10-fold. This also helps to reduce the potential for expression of the target protein prior to induction, which for some target proteins, have deleterious effects on host cell growth, thus, reducing maximum target protein production. The lactose repressor is produced from an endogenous host cell gene called lacI. However, host strains with lacI gene cannot produce enough lactose repressor to effectively repress expression of the target protein. Thus, to obtain the appropriate regulation of target protein the host strain should also contain an extra lacI-gene or use an overexpressing host cell comprising a lacI^{Q1} promoter.

Probably the single most advantageous characteristic of the expression system is the fact the T7 RNA polymerase is nearly 12-fold more processive than the host cell RNA polymerase. The high processivity of the T7 RNA polymerase can generate more than 60% of the cell's total protein as the target protein, making it among the most efficient expression systems available.

The basis for the present invention, however, is the discovery that in instances where the target protein is produced in large quantities, infectious phage is detectable in the fermentation broth. This suggests that the DE3 phage has regained its ability to be lytic. The high cell densities achieved during fermentation may be such that the infectious phage is generated through low levels of recombination or illegitimate recombination (reversion) resulting in excision of the lysogen. Nonetheless, regulatory agencies prohibit forward processing of a fermentation broth that contains target proteins to be used as pharmaceuticals that have detectable levels of phage particles.

In light of this problem, the present invention provides an improved T7 expression system. In the present invention, the T7 RNA polymerase gene is integrated into the chromosome of the host cell using a different integration mechanism. The present invention integrates a copy of the T7 RNA polymerase gene into a nonessential site in the chromosome of the host cell by homologous recombination instead of infecting the host cell with defective phage. The host cell further comprises a nonintegrated gene encoding a target protein of choice. The integrated gene encoding the T7 RNA polymerase is under control an endogenous regulatory system of the host cell, while the nonintegrated gene encoding the target protein is under control of a phage regulatory system. When the host cell is induced, a host cell RNA polymerase is able to bind to a host cell promoter and initiate synthesis of the T7 RNA polymerase. The newly synthesized T7 RNA polymerase is available to bind to a T7 or T7lac promoter and initiate synthesis of the target protein. The result is a phage-free fermentation broth comprising the target protein.

The present invention provides a host cell comprising a homologously recombinated T7 RNA polymerase gene under control of a lac promoter integrated into the host chromosome. The T7 RNA polymerase is integrated into the host cell chromosome without the use of a phage lysogen, resulting in no incorporation of additional phage DNA. Homologous recombination can occur in any nonessential gene of choice, whereas the phage lysogen integrates only at sites driven by the infection process. The promoter can be the wild type lac promoter or a modified lac promoter like, lacUV5.

The host cell can further comprise a nonintegrated gene encoding a target protein wherein the nonintegrated gene is under control of a T7 or T7lac promoter. Preferably the T7 promoter is T7lac. Preferably the target protein is parathyroid hormone (PTH) (1-84) or active fragments thereof, including N-terminal fragment 1-34, 1-31, 1-28, or analogs or derivatives thereof. In another embodiment, the target protein is glucagon-like peptide-1 (GLP-1), or analogs or derivatives thereof.

The present invention further provides an expression system for producing phage-free fermentation broth comprising a target protein, wherein the expression system comprises a host cell with a homologously integrated T7 RNA polymerase gene in a nonessential gene on a chromosome of a host cell and a nonintegrated gene encoding the target protein.

The present invention further provides a process for preparing a host cell comprising a homologously integrated T7 RNA polymerase gene. The T7 RNA polymerase gene is integrated into any nonessential gene of the host chromosome, preferably, the galactose operon of the host chromosome. The T7 RNA polymerase gene can be integrated into the galactose operon from a plasmid selected from the group consisting of pHMM209, pHMM220, pHMM223, and pHMM228.

The present invention further provides a process for preparing a target protein which comprises expressing the target protein in a host cell comprising a homologously integrated T7 RNA polymerase gene, and wherein the target protein is phage-free. Preferably the target protein is parathyroid hormone (PTH) (1-84) or active fragments thereof, including N-terminal fragment 1-34, 1-31, 1-28, or analogs or derivatives thereof. In another embodiment, the target protein is glucagon-like peptide-1 (GLP-1), or analogs or derivatives thereof.

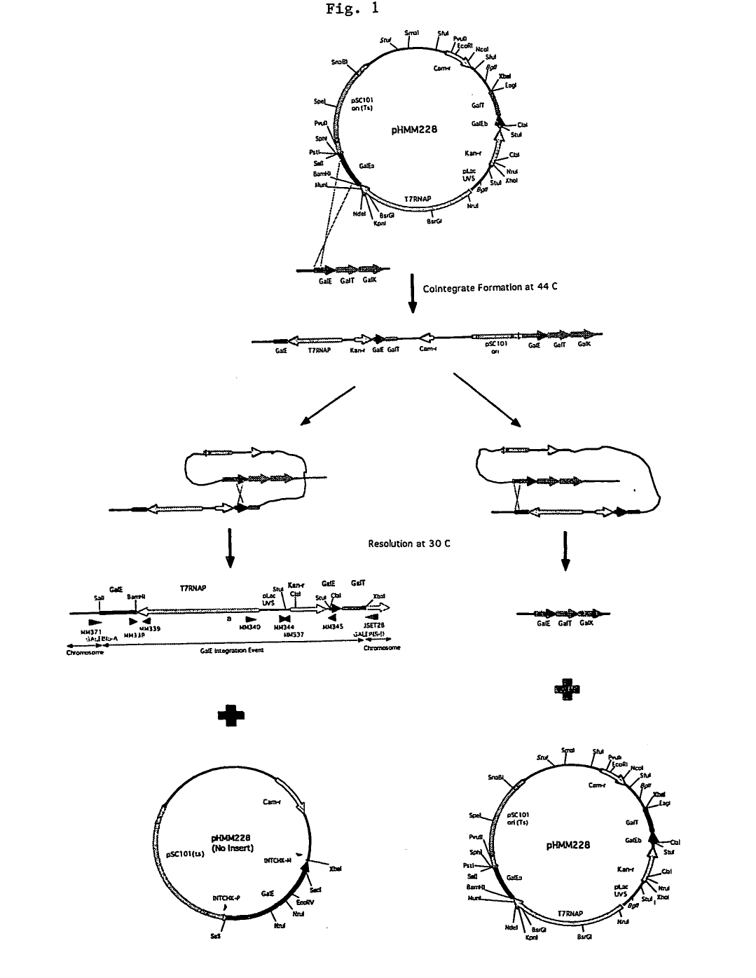

FIG. 1 shows a schematic representation of homologous recombination of the T7 RNA polymerase from the integration plasmid pHMM228 into the host chromosome.

For purposes of the present invention, as disclosed and claimed herein, the following general molecular biology terms and abbreviations are defined below. The terms and abbreviations used in this document have their normal meanings unless otherwise designated. Amino acids abbreviations are as set forth in 37 C.F.R. § 1.822 (b)(2) (1994).

“Base pair” or “bp” as used herein refers to DNA. The abbreviations A,C,G, and T correspond to the 5′-monophosphate forms of the deoxyribonucleosides (deoxy)adenosine, (deoxy)cytidine, (deoxy)guanosine, and thymidine, respectively, when they occur in DNA molecules. In double stranded DNA, base pair may refer to a partnership of A with T or C with G. “Kilo-base” or “kb” refers to one thousand (1000) base pairs.

“Plasmid” refers to an extrachromosomal genetic element comprising nucleic acid. Plasmids are generally designated by a lower case “p” followed by letters and/or numbers. The starting plasmids herein are either commercially available, publicly available on an unrestricted basis, or can be constructed from available plasmids in accordance with published procedures. In addition, equivalent plasmids to those described are known in the art and will be apparent to the ordinarily skilled artisan. Plasmids comprise DNA molecules to which one or more additional DNA segments can or have been added. Some plasmids are temperature sensitive while others are not. This means that at permissive temperatures, some plasmids are self-replicating, and at nonpermissive temperatures, some plasmids are not self-replicating.

“Expression plasmid” as used herein refers to any nontemperature sensitive plasmid in which a promoter to control transcription of the inserted DNA has been incorporated. A T7 expression plasmid comprises a T7 or T7lac promoter that controls expression of a target gene encoding a target protein. T7 expression plasmids are well known to the ordinarily skilled artisan. T7 expression plasmids are commercially available from Novagen, Inc. (Madison Wis.), and include but are not limited to the pET series of expression plasmids.

“Integration plasmid” as used herein refers to any temperature sensitive plasmid in which a promoter to control transcription of the inserted DNA has been incorporated. Additionally, the integration plasmid inserts a specified segment of DNA into the chromosome of a cell. Integration plasmids are derived from pMAK700 and pMAK705. The pMAK700 and pMAK705 are generated as described by Hamilton, et al., J. Bacteriol. 171:4617-4622, (1989) which is herein incorporated by reference in its entirety. Integration plasmids of the present invention, pHMM228, pHMM209, pHMM220, and pHMM223 are described in detail below. These integration plasmids comprise a lac promoter that controls expression of the T7 RNA polymerase gene encoding the T7 RNA polymerase.

“Transformation” refers to the introduction of a plasmid into an organism so that the plasmid is replicable, either as an extrachromosomal element or by chromosomal integration. Methods of transforming bacterial and eukaryotic hosts are well known in the art, many of which methods are summarized in J. Sambrook, et al., Molecular Cloning: A Laboratory Manual, (1989). Successful transformation is generally recognized when any indication of the operation of this plasmid occurs within the host cell. For example, a sensitive host cell will become resistant to a selecting agent when the host cell is transfected with a plasmid that allows for the resistance.

“Permissive temperature” is the temperature which a plasmid after transformation into the host cell can self replicate independent of cell duplication. The permissive temperature as defined in this invention is a temperature typically less than 44° C., generally between about 20° C. and about 40° C., preferably between about 25° C. and 40° C., more preferably between about 25° C. and 35° C., most preferably about 30° C.

“Nonpermissive temperature” is the temperature which a plasmid after transformation into the host cell cannot self replicate independent of cell duplication. The nonpermissive temperature as defined in this invention is a temperature typically greater than 40° C., generally between about 40° C. and about 50° C., preferably about 44° C.

“Transcription” refers to the process whereby information contained in a nucleotide sequence of DNA is transferred to a complementary RNA sequence by RNA polymerase. For example E. coli RNA polymerase transfers the T7 RNA polymerase gene to the complementary RNA sequence which is then translated into T7 RNA polymerase. Likewise, for example, T7 RNA polymerase transfers the target gene to the complementary RNA sequence which is then translated into the target protein.

“Translation” as used herein refers to the process whereby the genetic information of messenger RNA (mRNA) is used to specify and direct the synthesis of a polypeptide chain.

“Isolated amino acid sequence” refers to any amino acid sequence, however, constructed or synthesized, which is locationally distinct from the naturally occurring sequence.

“Isolated DNA compound” refers to any DNA sequence, however constructed or synthesized, which is locationally distinct from its natural location in genomic DNA.

“Promoter” refers to a DNA sequence which binds an RNA polymerase and directs transcription of DNA to RNA. Example of promoters used herein are lac, lacUV5, T7, T7lac, lacI^{Q1}.

“PCR” refers to the widely-known polymerase chain reaction employing a thermally-stable DNA polymerase.

“Primer” refers to a nucleic acid fragment which functions as an initiating substrate for enzymatic or synthetic elongation in PCR.

“Parental cell” refers to a cell that is void of a lysogen and is capable of self-replicating in vitro. The parental cell should also have DNA sequences that are determinable and should be approximately 2 kb in length of the host cell chromosome. These sequences should further be in a nonessential area of the cell. Preferably, the parental cell is bacterial. Preferably, the parental cell comprises DNA sequences of the galactose operon or a segment thereof. Preferably, the parental cell is E. coli. Preferred E. coli parental cells are commercially available from several suppliers such as Novagen, Inc. (Madison Wis.), and include but are not limited to BL21, AD494, BLR, HMS174, Origami, and Tuner.

“Host cell” in the present invention refers to a parental cell that comprises a homologously integrated T7 RNA polymerase gene under control of a lac promoter. The promoter can be the wild type lac promoter or a modified lac promoter like lacUV5. The host cell can further comprise a nonintegrated gene under control of a T7 promoter. The promoter can be the wild type T7 promoter or a modified T7 promoter like T7lac. The nonintegrated gene encodes a target protein of choice. Upon induction of the host cell, T7 RNA polymerase is produced. The T7 RNA polymerase is then available to produce the target protein in phage-free fermentation broth.

“Phage-free” refers to no observable plaques on a lawn of bacteria when incubated with fermentation broth. Assays used to test for phage contamination are well known in the art.

“Homologously integrated gene” refers to a gene that is integrated into the chromosome of a host cell by a method of homologous recombination. The method of homologous recombination proceeds between a DNA sequence on the chromosome of the host cell and complementary sequences carried on an integration plasmid that is present inside the cell after transformation. Preferably, the method of homologous recombination is performed as taught by Hamilton, et al. in New method for generating deletions and gene replacements in Escherichia coli. J. Bacteriol. 171:4617-4622, 1989, which is herein incorporated by reference.

“Complementary” as used herein, refers to pairs of bases (purines and pyrimidines) that associate through hydrogen bonding in a double stranded nucleic acid. The following base pairs are complementary: guanine and cytosine; adenine and thymine; and adenine and uracil.

The gene that is integrated by homologous recombination in accordance with the present invention is a T7 RNA polymerase gene. The T7 RNA polymerase gene is obtained from T7 bacteriophage and is under control of an isopropylthio-β-galactoside (IPTG) inducible lacUV5 promoter. The gene can be obtained from plasmid pAR1219, American Type Culture Collection (ATCC) 39563, U.S. Pat. No. 4,952,496. A BamHI fragment in pAR1219 contains a T7 expression cassette comprising a T7 RNA polymerase gene under control of the IPTG-inducible lacUV5 promoter, and a lacI gene under control of the its native promoter.

The T7 RNA polymerase gene encodes a T7 RNA polymerase that is well known in the art and is described in detail in U.S. Pat. No. 4,952,496, which is herein incorporated by reference. When the host cell is induced, a host cell RNA polymerase is able to bind to the lacUV5 promoter and initiate synthesis of the T7 RNA polymerase.

“Nonintegrated gene” refers to a gene that is not integrated into the chromosome of a host cell, but is carried in an expression plasmid. The expression plasmid is introduced into the host cell by routine and conventional transformation methods, and replicates autonomously within the host cell at permissive temperatures. Thus, the plasmid can replicate itself in the host cell in the absence of host cell duplication. The nonintegrated gene that is carried in the expression plasmid encodes a target protein of interest. The nonintegrated gene is under control of an isopropylthio-β-galactoside (IFFG) inducible T7 or T7lac promoter. The newly synthesized T7 RNA polymerase from the integrated gene is able to bind to the T7 or T7lac promoter and initiate synthesis of the target protein.

“Target protein” refers to a protein that can be synthesized in a host cell. Preferably the target protein is heterologous to host cell proteins. Examples of proteins include but are not limited to calcitonin, erythropoietin (EPO), factor IX, factor VIII, granulocyte colony stimulating factor (G-CSF), granulocyte macrophage colony stimulating factor (GM-CSF), macrophage colony stimulating factor (M-CSF), chemokines, growth hormone releasing factor (GRF), insulin-like growth factor (IGF-1), growth hormone, insulin, leptin, interferon, interleukins, luteinizing hormone releasing hormone (LHRH), follicle stimulating hormone (FSH), somatostatin, vasopressin, amylin, glucagon-like-peptide-1 (GLP-1), parathyroid hormone (PTH), exendin-3, exendin4, and alpha-1 anti-trypsin. The target protein of the present invention can optionally be a precursor protein or pro-protein. Examples of precursor proteins or pro-proteins include but are not limited to proinsulin and GIP-1(1-37).

Integration Construct:

Useful plasmids are constructed to allow the integration of the recombinant target gene into the chromosome of a desired host cell by homologous recombination. This integration can be accomplished using modified pMAK constructs. Preferably the starting pMAK constructs are pMAK700 and pMAK705. More preferably the starting pMAK construct is pMAK705. pMAK constructs comprise a temperature sensitive origin of replication. This allows the construct to replicate at permissive temperatures like 30° C., but the construct will not replicate at nonpermissive temperatures like 44° C. The pMAK constructs also comprise a chloramphenicol resistance (Cm^{r}) gene. Thus, a host cell that contains a plasmid that comprises a Cm^{r} gene will be resistant to chlorampehicol, and at a permissive temperature will replicate in the presence of chloramphenicol.

The pMAK constructs are modified by the insertion of nucleic acid sequences into the pMAK construct that are homologous to a nucleic acid sequence found on the chromosome of a host cell. pMAK constructs which are inserted with homologous nucleic acid sequences found on the chromosome of a host cell are referred to in the present invention as pHMM constructs. The homologous sequences of the pHMM constructs comprise different fragments of the galactose operon (galETK). The galactose operon is well known in the art. The homologous sequences of the pHMM construct and the host cell have sufficient length to hybridize to each other and undergo recombination. The hybridization generally depends on the ability of denatured chromosomal DNA to re-anneal when complementary strands from the integration construct are present in an environment like a host cell. Preferably the homologous sequence is greater than about 1 kb. More preferably, the homologous sequence is between about 1 kb and about 10 kb. Even more preferably, the homologous sequence is between about 1 kb and about 4 kb. Most preferably, the homologous sequence is about 2 kb. The homologous sequences of the host cell can be any sequence that is not essential to the host cell because the recombination event can disrupt the sequence such that the sequence can become nonuseful. For example, if the homologous sequence is in the gene responsible for the synthesis of the cell wall, recombination in this sequence of the host cell with the integration plasmid could disrupt synthesis of the proteins comprising the cell wall and result in a nonviable host cell.

The pHMM constructs can be further modified by the insertion of a T7 RNA polymerase gene and a lacUV5 promoter into the pHMM construct. A T7 RNA polymerase gene under control of the lacUV5 promoter can be obtained from plasmid pAR1219, American Type Culture Collection (ATCC) 39563, U.S. Pat. No. 4,952,496.

Preferably, the original lac promoter of the pMAK plasmid is eliminated by the cloning of the T7 RNA polymerase gene and the lacUV5 promoter into the pHMM construct. A duplication of lac promoters could result in the potential for secondary structure formation, which might present problems for sequence determination and the possibility for interfering with homologous recombination.

Optionally, a pHMM construct can be further modified by the insertion of a lacI gene into the pHMM construct. In addition to the T7 RNA polymerase gene and the lacUV5 promoter, the pAR1219 plasmid further comprises a DNA fragment containing the lacI gene under control of its native promoter. A copy of the lacI gene in the expression system can provide additional expression of the lactose repressor which helps control both T7 RNA polymerase and target protein expression.

Optionally, the lacI gene from the T7 expression cassette driven by a lacI^{Q1} promoter. The lacI^{Q1} promoter is well known in the art. The lacI^{Q1} promoter is modified to overexpress the lad gene. The result is about 100× production of the lacI repressor than the lacI gene driven by its native promoter.

Additionally, a pHMM construct further comprises a second resistance gene. Preferably the second resistance gene is kanamycin (Km^{r}). Thus, a host cell that contains a plasmid that comprises a Km^{r} gene will be resistant to kanamycin and will replicate in the presence of kanamycin. Preferably, the second resistance gene is oriented in the opposite direction as the T7 RNA polymerase. The kanamycin resistance gene provides an additional means of uniquely identifying the host cell. The kanamycin resistance gene can be obtained from the plasmid pACYC177. pACYC177 is available from “Stratagene Cloning Systems” Catalog (1993) (Stratagene, La Jolla, Calif.). The kanamycin resistance gene from pACYC177 includes Tn903 transposition inverted repeats (IR). Due to potential instability through transposition resulting from the presence of these inverted repeats, a cassette encompassing the kanamycin resistance gene but not the inverted repeat sequences is preferred.

Integration:

An integration construct can be transformed into a desired host strain according to conventional methods and individual colonies are grown overnight in liquid growth media at permissive temperature in the presence of a selection agent, for example, Cm or Km. The resulting overnight culture is diluted in liquid growth media in the presence of a selecting agent and incubated at a nonpermissive temperature, for example, 44° C., until log phase. The culture is then plated on agar plates containing a selection agent and incubated overnight at nonpermissive temperature to select for cointegrate formation. Cointegrate formation is the initial step in homologous recombination and occurs when the integration construct integrates into the host chromosome. Because the integration plasmid cannot replicate itself at a nonpermissive temperature and the culture contains a selecting agent, the only host cells that will survive under these conditions will be those that integrate the integration construct into the host cell chromosome. The resulting culture is plated on agar plates comprising a selecting agent and incubated at nonpermissive temperature overnight to select for cointegrates.

A pool of cointegrate colonies are picked, transferred to liquid growth media, and incubated overnight at permissive temperature for resolution of the cointegrate. Resolution provides a means for a second recombination event to occur whereby the integration plasmid is excised from the chromosome and reformed within the host cell. The integration plasmid that is excised and reformed in the host cell is either the original integration plasmid in whole or is the original integration plasmid minus the T7 RNA polymerase which remains integrated into the chromosome of the host cell. The objective of the second recombination event is to excise the portion of the integration plasmid that comprises the origin of replication from the host cell chromosome, but to leave the T7 RNA polymerase integrated into the chromosome of the host cell. A schematic of this process is shown in FIG. 1. In the cases where the integration plasmid further comprises other genes, for example lacI or Km, the objective of the second recombination event is to excise the portion of the integration plasmid that comprises the origin of replication from the host cell chromosome, but to leave the T7 RNA polymerase, and other genes, for example lacI or Km, integrated into the chromosome of the host cell. Removal of the origin of replication of the integration plasmid is desired because an integrated origin of replication could be deleterious to the host cell. This excision process may optionally be continued for days by subculturing with a selecting agent and maintaining at permissive temperature. Preferably, subculturing and maintaining is less than three days, more preferably subculturing and maintaining is continued for two days.

The culture is then diluted into a pre-warmed flask contain liquid growth media without a selecting agent at nonpermissive temperature to initiate curing of the integrate by excising undesirable plasmid sequence from the chromosome of the host cell. The culture is plated on agar plates containing a selection agent and grown at permissive temperature. Colonies are screened for the presence of an integration event using means known to a skilled artisan, for example, PCR and Southern blotting. Colonies containing an integrate are used to inoculate a liquid media culture and subsequently grown for consecutive days at nonpermissive temperature to promote curing. The cultures and can then plated onto agar plates and incubated overnight at permissive temperature. Individual colonies can subsequently be patched onto agar plates optionally containing both selecting agents, for example Cm and Km. The individual colonies can further be patched onto agar plates containing only the second selecting agent, for example Km. The desired clones which have integrated sequences are Cm sensitive and Km resistant.

In another embodiment, the integration plasmid is preferably integrated into the galactose operon of the host cell. More preferably, the integration plasmid is integrated into the galE locus of the host cell. Several attempts were made to integrate into the galK locus, however ideal integration was unsuccessful.

Target Protein:

The nonintegrated gene encoding a recombinant target protein used in the expression system of the present invention is obtained by means available to ordinarily skilled artisans in the field of molecular biology. The basic steps are:
a) isolating a natural DNA sequence or constructing a synthetic or semi-synthetic DNA sequence, wherein either DNA sequence comprises a target gene that encodes a target protein of interest,
b) cloning the DNA sequence into an available T7 expression plasmid in a manner suitable for expressing the target protein,
c) transforming the previously described expression host of the present invention with the T7 expression plasmid comprising the target gene of interest,
d) culturing the transformed expression host for a period of time in an uninduced state and then for a period of time in an induced state, and
e) recovering and purifying the target protein.

Preferably, the target protein is parathyroid hormone (PTH). More preferably, the PTH is human PTH. PTH is known in the art as an 84 amino acid protein and described in U.S. Pat. No. 5,496,801. N-terminal fragments of PTH are also well known in the art and include but are not limited to 1-34, 1-31, and 1-28. Also, contemplated are analogs and derivatives of PTH and PITH fragments. Examples of PTH fragments, analogs and derivatives are described in WO99/29337, U.S. Ser. No. 20020132973, U.S. Pat. Nos. 5,556,940; 6,472,505; and 6,417,333.

In another embodiment, the target protein is glucagon-like peptide-1 (GLP-1), or analogs or derivatives thereof. Examples of GLP-1 analogs and derivatives are well known in the art and are described in WO01/98331, and U.S. Pat. Nos. 6,268,343; 5,977,071; 5,545,618; 5,705,483; and 6,133,235. GLP-1 analogs also include Exendin-3 and Exendin-4 agonists as described in WO99/07404, WO99/25727, WO99125728, WO99/43708, WO00/66629, and U.S. Ser. No. 2001/0047084A1.

Modification:

The isolated target protein is useful as a therapeutic protein. Optionally the target protein can be further modified outside the host cell to give the target protein additional physical characteristics useful for a therapeutic protein. Modifications include but are not limited to enzymatic or chemical cleavages, acylation, crystallization, salt additions, and the like.

Prepartations:

Liquid growth media is T Broth

T Broth=(per liter) 10 g tryptone, 5 g yeast extract, 10 g NaCl, pH 7.5.

T agar plates=add 15 g/L agar to T broth.

SM buffer=(per 100 mL of 10× solution) 20 mL 1M Tris-HCl (pH 7.4), 20 mL 5M NaCl, 10 mL 1M MgSO₄

Chloramphenicol (Cm)(25 ug/mL) in ethanol.

Kanamycin (Kam)(15-50 ug/mL) in water.

Nalidixic acid (20 ug/mL) in NaOH

Streptomycin (50 ug/mL) in water

Integration Plasmid pHMM209:

The integration plasmid pHMM209 is a pMAK705 derivative. The initial step in the construction of pHMM209 is to clone an oligonucleotide adapter, BamHI to ClaI, into the pMAK705 backbone. This adapter contains a StuI site, which is unique in the resulting construct. A galK flank is cloned into the pMAK705 backbone as a SalI to XbaI insert resulting in a pHMM backbone. The pHMM backbone comprises unique BamHI and ClaI sites in the galK flank. The T7 expression cassette from pAR1219, comprising the lacI gene under the expression of its native promoter sequence and the T7 RNA polymerase gene under the regulation of the lacUV5 promoter is then cloned as a BamHI fragment into the pHMM backbone. The orientation of the T7 expression cassette is opposite that of the galETK operon to prevent transcriptional read-through from galE upstream sequences. Next, the resistance gene for kanamycin is cloned as a StuI fragment from pACYC177 into the StuI site of the adapter that was previously cloned into the pMAK705 backbone. The orientation of the kanamycin gene is opposite that of the T7 expression cassette. The resulting integration plasmid is pHMM209.

Integration Plasmid pHMM220:

The integration plasmid pHMM220 is a pMAK705 derivative. The initial step in the construction of pHMM220 is to clone an oligonucleotide adapter, BamHI to ClaI, into the pMAK705 backbone. This adapter contains a StuI site, which is unique in the resulting construct. A galK flank is cloned into the pMAK705 backbone as a SalI to XbaI insert resulting in a pHMM backbone. The pHMM backbone comprises in unique BamHI and ClaI sites in the galK flank. The T7 expression cassette, comprising the lacI gene under the expression of its native promoter sequence and the T7 RNA polymerase gene under the regulation of the lacUV5 promoter is then cloned as a BamHI fragment from pAR1219 into the pHMM backbone. The orientation of the T7 expression cassette is opposite that of the galETK operon to prevent transcriptional read-through from galE upstream sequences. Next, a kanamycin resistance gene as a StuI fragment is obtained by PCR. The PCR primers that are used to amplify the resistance gene are designed inside the inverted repeat sequences present in the pACYC177 template kanamycin gene. The PCR primers contain StuI restriction sites in their tails and they are used in an amplification reaction. The resulting approximately 1 kb PCR product is cloned directly into a PCR cloning plasmid and putative clones are selected for by plating directly on T agar plates containing kanamycin. The resulting kanamycin resistance gene is subcloned as a StuI fragment into the StuI site of the adapter that was previously cloned into the pMAK705 backbone. The orientation of the kanamycin gene is opposite that of the T7 expression cassette. The resulting integration plasmid is pHMM220.

Integration Plasmid pHMM223:

Integration plasmid pHMM223 is constructed the same as pHMM220. Next, the lad gene of the T7 expression cassette in pHMM220 is removed because the lacI gene had the potential to integrate into the lacI locus of the host chromosome. The lacI gene is deleted from the pHMM220 by digestion of the plasmid using BglI. A synthetic DNA adapter is cloned into the BglI site to reconstitute the lacUV5 promoter that is deleted in the BglI digestion process. The resulting clone is sequenced and is found to contain the desired lacUV5 sequence with the exception of two nucleotide changes. These changes are in the 5′ untranslated region of the T7 expression cassette and are not critical to expression of the T7 RNA polymerase. Next, the lacI promoter present in the pHMM220 is eliminated. This is accomplished by insertion of a PstI to AseI adapter that completely replaces the lacI promoter sequence.

The BglI deletion of the pHMM220 also removes the downstream galK flank. In order to reconstitute this region and incorporate the kanamycin resistance gene without the inverted repeats, a BamHI to XbaI fragment is subcloned into the BglII to XbaI sites of the integration plasmid. This results in an integration plasmid designated pHMM223, which contains the T7 expression cassette without a copy of the lacI gene and the lacI promoter, kanamycin resistance gene without inverted repeats, and a complete galK flank. The pHMM223 is used for attempts to integrate into the galK locus of the chromosome.

Integration Plasmid pHMM228:

The integration plasmid pHMM228 is a pMAK705 derivative. The initial step in the construction of pHMM228 is to clone an oligonucleotide adapter, PstI to EagI, into the pMAK705 backbone. This adapter contains unique SalI and XbaI sites. Approximately 2 kb of the galE gene is cloned into the pMAK705 backbone as a SalI to XbaI insert resulting in a pHMM backbone. The pHMM backbone comprises unique BamHI and ClaI sites in the gene. The T7 expression cassette, comprising the lacI gene under the expression of its native promoter sequence and the T7 RNA polymerase gene under the regulation of the lacUV5 promoter is then cloned as a BamHI fragment from pAR1219 into the pHMM backbone. The orientation of the T7 expression cassette is opposite that of the galETK operon to prevent transcriptional read-through from galE upstream sequences. Next, a kanamycin resistance gene as a StuI fragment is obtained by PCR. The PCR primers that are used to amplify the resistance gene are designed inside the inverted repeat sequences present in the pACYC177 template kanamycin gene. The PCR primers contain StuI restriction sites in their tails and they are used in an amplification reaction. The resulting approximately 1 kb PCR product is cloned directly into a PCR cloning plasmid and putative clones are selected for by plating directly on T agar plates containing kanamycin. The resulting kanamycin resistance gene is subcloned as a StuI fragment into the StuI site of the adapter that was previously cloned into the pMAK705 backbone. The orientation of the kanamycin gene is opposite that of the T7 expression cassette. Finally, the lacI gene of the T7 expression cassette is removed essentially as described for pHMM223. The lacI gene is deleted by digestion of the plasmid using BglI. A synthetic DNA adapter is cloned into the BglI site to reconstitute the lacUV5 promoter that is deleted in the BglI digestion process. Next, the lacI promoter is eliminated by insertion of a PstI to AseI adapter that completely replaces the lacI promoter sequence. The BglI deletion also removes the downstream galE flank. In order to reconstitute this region and incorporate the kanamycin resistance gene without the inverted repeats, a BamHI to XbaI fragment is subcloned into the BglII to XbaI sites of the integration plasmid. This results in an integration plasmid designated pHMM228, which contains the T7 expression cassette without a copy of the lacI gene and lacI promoter, kanamycin resistance gene without inverted repeats, and a complete galE flank. The pHMM228 is used for attempts to integrate into the galE locus of the chromosome.

Integration/Screening of pHMM209:

The integration plasmid pHMM209 is transformed into a E. coli parental cell line comprising a galactose operon, plated on T agar plates containing Cm, and incubated overnight at 30° C. Colonies where picked, transferred to T broth containing Cm and grown overnight at 30° C. The resulting overnight culture is diluted in T broth in the presence of Cm and incubated at 44° C., until the culture reaches log phase. The culture is then plated on T agar plates comprising Cm and incubated overnight at 44° C. to induce cointegrate formation. A pool of cointegrate colonies are picked, transferred to 250 mL of T broth containing Cm, and incubated overnight at 30° C. for excision and resolution. This culture is maintained for two more days by sub-culturing at a 1:500 dilution with T broth containing Cm and incubating the flask at 30° C. On the fourth day, the culture is sub-cultured into a pre-warmed flask of T broth at 44° C. This culture is grown and sub-cultured for three consecutive days at 44° C. to promote curing of the pHMM209 plasmid. The tentatively integrated, excised and cured culture is then plated onto T agar plates containing Km and incubated overnight at 30° C. Individual colonies are subsequently patched onto T agar plates containing Cm and Km, then onto T agar plates containing Km, then onto T agar plates. Positive integrates should be Cm^{s} and Km^{r}.

Nearly 1000 individual colonies were tested and only one integrate was formed. This integrate is designated RQ209. Further analyses showed that the RQ209 strain possessed functional T7 RNA polymerase that was induced by the addition of IPTG. However, when PCR mapping was performed on the RQ209 strain, it was found that the T7 RNA polymerase had not specifically integrated into the galK or the lacI regions of the chromosome.

Integration/Screening for pHMM228:

Integration experiments were carried out essential as described in integration/screening of pHMM209 above. The table below shows the number of cointegrates formed.

TABLE 1
Cointegrates of pHMM228

| | Plate Counts | | | | | | |
|---|---|---|---|---|---|---|---|
| | 10⁻¹ | 10⁻² | 10⁻³ | 10⁻⁴ | 10⁻⁵ | 10⁻⁶ | 10⁻⁷ |
| Plating | ND | ND | ND | TNTC | 73 | 8 | 1 |
| Temperature | | | | | | | |
| 30° C. | | | | | | | |
| Plating | TNTC | TNTC | TNTC | 153 | 11 | 0 | ND |
| Temperature | | | | | | | |
| 44° C. | | | | | | | |

TNTC: to numerous to count
ND: not determined

Colonies that grew on the 44° C. plates were subsequently grown in T broth at 44° C. Nine individual isolates were grown in addition to one culture that was pooled from colonies representing approximately ½ an entire plate. These 10 cultures were shaken (315 rpm) overnight under Cm selection at 44° C. The following day 100 uL samples from each were harvested by centrifugation for subsequent PCR analyses. In addition, plates pre-warmed to 44° C. were used to streak for individual isolates from these cultures and incubated overnight at 44° C. PCR and restriction mapping results showed that nearly all of the 10 liquid cultures contained an amplification product consistent with the expected integration event.

The individuals clones were screened by re-patching at 44° C. Individual isolate #2 was grown up in T broth containing Cm at 30° C. overnight to promote excision of pHMM228. After overnight growth, a 100 uL sample of cells were collected and used as template for a PCR reaction. Primers from outside the galE flanks were chosen so that the only amplification would be the excised version of pHMM228. Thus if the excision event regenerates pHMM228, an approximately 7 kb PCR product would be expected. However, if excision resulted from a second crossover event leaving the T7 RNA polymerase in the chromosome, an approximately 1.5 Kb PCR product would be expected. As expected, a mixture of excision products is observed. The excised culture is subsequently streaked out to obtain individual isolates which are screened for the presence of the 1.5 Kb PCR product.

Three isolates were then grown overnight without selection at 44° C. in T broth to promote curing of the excised pHMM228. After overnight growth at 44° C., single colonies were isolated from streaked T agar plates and 72 individuals from each of the three original isolates were patched onto T plates plus Cm, T plates plus Km, and T plates to determine those that had been successfully cured of the excised pHMM228. The table below details the results of these experiments.

TABLE 2
Curing Efficiency

| | Total | | | |
|---|---|---|---|---|
| | Individuals | | | Curing |
| Isolate | Analyzed | Cm^{r} | Km^{r} | Efficiency (%) |
| #1 | 72 | 24 | 72 | 66.7 |
| #6 | 72 | 37 | 72 | 48.6 |
| #15 | 72 | 14 | 56 | 56.9 |

A single Cm-sensitive individual designated RQ228 was subsequently chosen from the isolate #1 and was streaked for purification two times and phenotypically verified. The table below shows the results of the phenotypic analyses.

TABLE 3
Phenotypic Results

| | Result of |
|---|---|
| Phenotype Plate | Patching |
| M9 | no growth |
| M9 + galactose | no growth |
| M9 + lactose | growth |
| M9 + glucose | growth |
| L + Cm | no growth |
| L + streptomycin | growth |
| L + Km | growth |
| L + Nalidixic Acid | no growth |
| L | growth |

A colony of the RQ228 strain that was phenotype confirmed was then chosen and a 10 mL culture was grown up overnight for local as well as long-term preservation and used to make a competent cell lot. This same colony was used in integration integrity PCR mapping.

T7 Activity and Regulation Assay:

In addition to confirming the phenotypic characteristics and the integrity of the integration event, the RQ228 strain was also examined for its ability to express functional T7 RNA polymerase as well as the ability of this expression to be regulated. The ability of the RQ228 strain to rescue the defective T7 tester phage was examined as described below.

T7 RNA Polymerase Assay:

A T7 RNA polymerase activity assay was utilized in order to determine whether the RQ209 strain or the RQ228 strain possessed functional T7 RNA polymerase. The RQ209 strain or the RQ228 strain were grown at about 37° C. overnight in T broth supplemented with 0.2% maltose and 10 mM MgSO₄. Overnight cultures were diluted back to OD₆₀₀=0.05 in T broth again supplemented with 0.2% maltose and 10 mM MgSO₄ and grown shaking to OD₆₀₀=0.5 and 100 uL of each bacterial culture was added to 100 uL of a 10⁻⁶ dilution in SM buffer of the T7 tester phage. The samples were gently mixed by finger vortexing and incubated at 37° C. for 20 minutes to allow phage adsorption. Three mL of 0.4% T top agarose (supplemented with 0.2% maltose and 10 mM MgSO₄) was then added to the samples, vortexed and poured onto pre-warmed T agar plates. Each sample was prepared in duplicate so that one could be plated on T agar and the other could be plated on T agar containing 400 uM IPTG. The T7 tester phage can attach to the cells but can replicate and lyse only those cells it infects that have functional T7 RNA polymerase available. Since T7 RNA polymerase expression is under the control of the lacUV5 promoter, expression should only result in presence of the inducer, IPTG. Plaques on the plate containing EPTG and no plaques on the plate without IPTG constitutes a positive indication of controlled expression of T7 RNA polymerase.

Final PCR Confirmation of Integration Integrity:

The RQ228 strain was analyzed to confirm the integrity/specificity of the integration event. PCR amplifications were performed to examine both junctions of the galE-targeted integration event as well as confirmation of the size of the entire integration cassette.

PTH Expression Using RQ228:

An expression plasmid comprising the gene for PTH is transformed into either of RQ228 or a DE3 host cell using conventional methods. Both strains are grown at 37° C. in T broth containing tetracycline and induced by the addition of IPTG to 10 uM. The cultures are continued to incubate for 6 hours. Samples of the cultures show that both host cells express PTH. However, only the PTH produced in the RQ228 host cell is phage-free, while the PTH produced in the DE3 host cell has measurable levels of phage contamination in the fermentation broth.

E. coli is grown to saturation at 37° C. overnight in T broth supplemented with 0.2% maltose and 10 mM MgSO₄. The overnight culture is diluted back to OD₆₀₀=0.05 in T broth again supplemented with 0.2% maltose and 10 mM MgSO₄ and grown shaking to. OD₆₀₀=0.5 and 100 uL of E. coli culture is added to 100 uL of fermentation broth. The sample is gently mixed by finger vortexing and incubated at 37° C. for 20 minutes to allow phage adsorption. Three mL of 0.4% T top agarose (supplemented with 0.2% maltose and 10 mM MgSO₄) is added to the sample, vortexed and poured onto pre-warmed T agar plates. The plates are incubated at 37° C. for about 12 hours. Phage-free fermentation broth will produce no observable plaques.

1. A host cell comprising a homologously integrated T7 RNA polymerase gene under control of a lac promoter.

2. The host cell of claim 1 wherein the T7 RNA polymerase is integrated into a host cell chromosome without the use of a phage lysogen.

3. The host cell of claim 2 wherein the lac promoter is lacUV5 promoter.

4. The host cell of claim 2 or 3 wherein the T7 RNA polymerase gene is integrated into the galactose operon of the host chromosome.

5. The host cell of claim 4 wherein the T7 RNA polymerase gene is integrated into the galactose operon from an integration plasmid selected from the group consisting of pHMM209, pHMM22, pHMM223 and pHMM228.

6. The host cell of any one of claims 2 to 5 wherein the host cell further comprises a nonintegrated gene encoding a target protein under control of a T7lac promoter.

7. The host cell of claim 6 wherein the target protein is parathyroid hormone (PTH).

8. The host cell of claim 7 wherein the PTH is an N-terminal fragment of 1-84.

9. The host cell of claim 8 wherein the N-terminal fragment is 1-34.

10. The host cell of claim 6 wherein the target protein is glucagon-like peptide-1 (GLP-1), or a GLP-1 analog or derivative.

11. An expression system for producing a target protein in phage-free fermentation broth, wherein the expression system comprises a host cell with a homologously integrated T7 RNA polymerase gene in a nonessential gene of a host cell and a nonintegrated gene encoding the target protein, and wherein the nonintegrated gene is under control of a T7lac promoter.

12. The expression system of claim 11 wherein the T7 RNA polymerase gene is integrated into the galactose operon of the host chromosome.

13. The expression system of claim 12 wherein the T7 RNA polymerase gene is integrated into the galactose operon from an integration plasmid selected from the group consisting of pHMM209, pHMM22, pHMM223 and pHMM228.

14. The expression system of claim 13 wherein the target protein is parathyroid hormone (PTH).

15. The expression system of claim 14 wherein the PTH is an N-terminal fragment of 1-84.

16. The expression system of claim 15 wherein the N-terminal fragment is 1-34.

17. The expression system of claim 13 wherein the target protein is glucagon-like peptide-1 (GLP-1), or a GLP-1 analog or derivative.

18. A process of preparing a host cell comprising homologously integrating a T7 RNA polymerase gene under control of a lacUV5 promoter into a nonessential gene of the host, such that upon induction of the T7 RNA polymerase gene the fermentation broth will be phage-free.

19. The process of claim 18 wherein the T7 RNA polymerase gene is integrated into the galactose operon.

20. The process of claim 19 wherein the T7 RNA polymerase gene is integrated into the galactose operon from an integration plasmid selected from the group consisting of pHMM209, pHMM22, pHMM223 and pHMM228.

21. A process for preparing a target protein which comprises
a) preparing a host cell comprising homologously integrating a T7 RNA polymerase gene under control of a lacUV5 promoter into a nonessential gene of the host,
b) transforming the host cell with a nonintegrated gene encoding a target protein, and wherein the nonintegrated gene is under control of a T7lac promoter,
c) inducing the host cell to produce T7 RNA polymerase,
d) incubating the host cell in fermentation broth for a time sufficient to allow the T7 RNA polymerase to produce the target protein, and wherein the fermentation broth will be phage-free
